(19) United States
(12) Patent Application Publication
(10) Pub. No.: US 2021/0252191 A1
(43) Pub. Date: Aug. 19, 2021

(54) INJECTABLE AMNION HYDROGEL AS A CELL DELIVERY SYSTEM

(71) Applicant: UNIVERSITY OF CONNECTICUT, Farmington, CT (US)

(72) Inventors: Cato T. LAURENCIN, Avon, CT (US); Lakshmi S. NAIR, Avon, CT (US); Maumita BHATTACHARJEE, Farmington, CT (US)

(21) Appl. No.: 16/973,198

(22) PCT Filed: Jun. 11, 2019
(86) PCT No.: PCT/US2019/036476

§ 371 Date: Dec. 8, 2020

Related U.S. Application Data
(60) Provisional application No. 62/683,590, filed on Jun. 11, 2018.

(51) Int. Cl. A61L 27/36; A61L 27/38; A61L 27/52

(52) U.S. Cl. CPC A61L 27/3604; A61L 27/3687; A61L 27/3691; A61L 27/3695; A61L 2430/24; A61L 27/52; A61L 2400/06; A61L 2430/06; A61L 2430/16; A61L 27/3834

(57) ABSTRACT

Compositions including a decellularized and enzymatically solubilized amnionic membrane (AM) hydrogel, methods for making such compositions, and methods for their use are disclosed.

### CROSS REFERENCE

This application claims priority to U.S. Provisional Patent Application Ser. No. 62/683,590 filed Jun. 11, 2018, incorporated by reference herein in its entirety.

### STATEMENT OF GOVERNMENT RIGHTS

This invention was made with government support under Grant No. AR068147 awarded by the National Institutes of Health. The government has certain rights in the invention.

### BACKGROUND

Osteoarthritis (OA) is the most common degenerative joint disease and the leading cause of disability worldwide. With the growing population, it has been estimated that >20% of the population will be suffering from OA with more than 40 million people severely disabled by 2050. OA results from the interaction of various factors such as aging, genetic factors, body mass index, mechanical injury. Although OA is known to be primarily a disease of the cartilage, but other tissues including synovial membrane, and subchondral bone are also involved in mediating joint destruction. OA arises from the cartilage matrix wear and tear followed by the inflammatory processes which affect the joint surroundings. Recent studies have shown that inflammation and its induced catabolism plays an important role in promoting OA disease symptoms and accelerating the disease.

### SUMMARY

In a first aspect the disclosure provides decellularized amnionic membrane (AM) hydrogels, or precursors thereof. in one embodiment, no detectable exogenous polymer is present in the hydrogel. In another embodiment, the AM is present at between about 1 mg/ml and about 15 mg/ml, about 1 mg/ml and about 14 mg/ml, about 1 mg/ml and about 13 mg/ml, about 1 mg/ml and about 12 mg/ml, about 1 mg/ml and about 11 mg/ml, about 1 mg/ml and about 10 about 1 mg/ml and about 9 about 1 mg/ml and about 8 mg/ml, about 2 mg/ml and about 15 mg/ml, about 2 mg/ml and about 14 mg/ml, about 2 mg/ml and about 13 mg/ml, about 2 mg/ml and about 12 mg/ml, about 2 mg/ml and about 11 mg/ml, about 2 mg/ml and about 10 mg/ml, about 2 mg/ml and about 9 mg/ml, or about 2 mg/ml and about 8 mg/ml in the hydrogel or precursor thereof. In a further embodiment, the hydrogel has a swelling ratio of between about 5% and about 15%. In one embodiment, the hydrogel has a storage modulus of about 100 Pa to about 10,000 Pa and/or has shear-thinning properties.

In another embodiment, the AM hydrogel or precursor thereof further comprises biological cells within the hydrogel or precursor thereof. In one embodiment, the biological cells comprise stem cells, including but not limited to human or animal adult stem cells, embryonic stem cells and induced pluripotent stein cells. In another embodiment, the stem cells comprise mesenchymal stein cells or adipose-derived stem cells. In a further embodiment, the biological cells are present at a concentration of between about 1×10⁵ cells/ml and about 1×10⁸ cells/ml, about 1×10⁵ cells/ml and about 5×10⁷ cells/ml, or about 1×10⁵ cells/ml and about 1×10⁷ cells/ml. In one embodiment, the biological cells comprise human cells.

In one embodiment, the disclosure provides pharmaceutical compositions comprising:

(a) the AM hydrogel or precursor thereof of embodiment or combination of embodiments of the disclosure; and

(b) a pharmaceutically acceptable carrier.

In another embodiment, the composition is present within an injection device or a catheter.

In another aspect, the disclosure provides methods for treating a disorder, comprising administering to a subject in need thereof and amount effective to treat the disorder of the AM hydrogel or precursor thereof, or the pharmaceutical composition, of embodiment or combination of embodiments of the disclosure. In one embodiment, the disorder is selected from the group consisting of an inflammatory disease, inflanunatory and degenerative conditions of the soft tissues and joints, a musculoskeletal tissue order, a skin tissue disorder including but not limited to burns, wounds, and ulcers; and an eve disorder including but not limited to a corneal defect. In another embodiment, the disorder comprises an inflammatory: and/or degenerative conditions of the soft tissues or joints including but not limited to plantar fasciitis, achilles tendinosis, joint tendinitis, tennis/golfer's elbow, ligament damage, arthritis including but not limited to osteoarthritis and rheumatoid arthritis, rotator cuff inflammation and/or degeneration, and discogenic pain. In a further embodiment, the AM hydrogel or precursor thereof, or the pharmaceutical composition, is administered by injection, including but not limited to injection at a joint.

In a further aspect, the disclosure provides methods for preparing a decellularized amnionic membrane (AM) hydrogel, comprising:

(a) decellularizing amniotic membrane by application of a strong base, including but not limited to NaOH to produce decellularized AM;

(b) enzymatically solubilizing the decellularized AM to produce a decellularized and enzymatically solubilized amnionic AM;

(c) diluting the decellularized and enzymatically solubilized amnionic AM to a desired concentration and pH in buffer to produce a decellularized and enzymatically solubilized amnionic AM solution; and

(d) heating the decellularized and enzymatically solubilized amnionic AM solution to form a decellularized and enzymatically solubilized amnionic AM hydrogel.

In one embodiment, the strong base is applied at a concentration of about 0.1M to about 0.5M. In another embodiment, the method further comprises lyophilizing the decellularized AM prior to step (b). In a further embodiment, the enzymatically solubilizing comprises contacting decellularized AM with pepsin under conditions and for a time suitable to promote enzymatic solubilization of the decellularized AM. In one embodiment, the heating comprises heating the decellularized and enzymatically solubilized amnionic AM at between about 20° C. and about 40° C., between about 20° C. and about 37° C., between about 25° C. and about 40° C., between about 25° C. and about 37° C., or about 37° C. for a time sufficient to form the hydrogel. In another embodiment, the AM is present at between about 1 mg/ml and about 15 mg/ml, about 1 mg/ml and about 14 mg/ml, about 1 mg/ml and about 13 mg/ml, about 1 mg/ml and about 12 mg/ml, about 1 mg/ml and about 11 mg/ml, about 1 mg/ml and about 10 mg/ml, about 1 mg/ml and about 9 mg/ml, about 1 mg/ml and about 8 mg/ml, about 2 mg/ml and about 15 mg/ml, about 2 mg/ml and about 14 mg/ml, about 2 mg/ml and about 13 mg/ml, about 2 mg/ml and about 12 mg/ml, about 2 mg/ml and about 11 mg/ml, about 2 mg/ml and about 10 mg/ml, about 2 mg/ml and about 9 mg/ml, or about 2 mg/ml and about 8 mg/ml in the hydrogel and/or the decellularized and enzytnatically solubilized amnionic AM solution. In one embodiment the methods comprise adding biological cells to the decellularized and enzymatically solubilized amnionic AM prior to step (d). In another embodiment, the biological cells comprise stem cells, including but not limited to human or animal adult stem cells, embryonic stem cells and induced pluripotent stem cells. In a further embodiment, the stem cells comprise mesenchymal stem cells or adipose-derived stem cells. In another embodiment, the biological cells are added at a concentration of between about 1×10⁵ cells/ml and about 1×10⁸ cells/ml, about 1×10⁵ cells/ml and about 5×10⁷cells/ml, or about 1×10⁵ cells/ml and about 1×10⁷cells/ml of the decellularized and enzymatically solubilized amnionic AM solution. In one embodiment, the biological cells comprise human cells.

### DESCRIPTION OF THE FIGURES

FIG. 1. (A) Graph of DNA quantification shows a significant reduction of DNA content the decellularized AM. (n=6, ***p<0.0001). (B) Graph of GAG content of native and decellularized amnion and (C) Graph of total collagen content of native and decellularized amnion.

FIG. 2, Schematic representation of AM gel preparation.

FIG. 3. (A) MTS assay showing cell viability in group1-group5. (B) NO assay showing the production of NO in group1-group5

FIG. 4. Gene expression analysis of various MMPs, ADAMTS5, IL6 and TIMP

FIG. 5. A) H&E staining, B) Safranin-O staining of sham (control) knee joints with saline injection, animals showed no inflammation and unaltered articular cartilage and subchondral bone.

FIG. 6. A) H&E staining, B) Safranin-O staining of animals injected with 500 U collagenase H showed inflammation and thinning of the articular cartilage.

FIG. 7. H&E staining of treated rat knee joints after 4 weeks (A) PBS treated group (B) ADSC treated group (C) AM gel treated group (I)) AM gel with ADSC treated group.

FIG. 8. Safranin-O staining of treated rat knee joints after 4 weeks (A) PBS treated group (B) ADSC treated group (C) AM gel treated group (D) AM gel with ADSC treated group.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology. Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al, 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the term about means +1-5% of the recited parameter.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In one aspect, the disclosure provides decellularized amnionic membrane (AM) hydrogels, and non-gelled precursors thereof.

As used herein, the amniotic membrane (AM) is the innermost layer of the placenta consisting of a thick basement membrane and an avascular stromal matrix. AM contains collagens (types I, III, IV, V and VI), fibronectin, laminin, proteoglycans and hyaluronam. AM has the potential to suppress the expression of potent pro-inflammatory cytokines, such as IL-1α and IL-1β, and decrease matrix metalioproteinase (MMP) levels through expression of natural MMP inhibitors present in the membrane. The AM may be from any suitable source, including but not limited to human placenta or any animal tissue source.

The AM hydrogel comprises a gel constructed from a network of AM polymers in a suitable aqueous medium, such as any neutral buffer, including but not limited to phosphate buffered saline (PBS). As disclosed herein, the AM hydrogels are capable of gelation at physiological pH and temperature and thus can form a gel upon injection to a subject, such as at a joint. The AM hydrogels or precursors thereof can be used, for example, in cell delivery and for tissue regeneration in a minimally invasive and cost-effective manner. In one non-limiting example, amnion hydrogel along with stem cells can reduce inflammation and slow down cartilage degradation in osteoarthritis patients (human or animal) and thus can have novel therapeutic potential.

As used herein, a precursor of the AM hydrogel is decellularized amnionic membrane or powder (or any embodiment or combination of embodiments disclosed herein) prior to aelation at physiological pH and temperature. The precursors therefore can be. for example, injected to a site in a subject at which it is desired for the hydrogel to form.

As used herein, “decellularized” means removal of at least 90%, 91%, 92%, 93%, 94%, 95%, 96%, 97%, 98%, 99%, or all cells present in the AM starting material. In one embodiment, the AM hydrogels of the disclosure include no detectable cells other than those exogenously added, as described below.

The hydrogels or non-powder precursors thereof further include at least a minimum content of an aqueous medium, such as water or any neutral buffer including but not limited to PBS. In some embodiments, the AM hydrogel or non-powder precursor comprises at least 50% by weight, at least 55%, or at least 60% by weight of an aqueous medium based upon the total weight of the hydrogel or non-powder precursor (e.g., with a maximum of 80% by weight). In particular, the aqueous medium can comprise about 50% to about 80%, about 55% to about 75%, or about 60% to about 70% by weight of the hydrogel based on the total weight of the hydrogel or non-powder precursor.

In one embodiment, no detectable exogenous polymer is present in the AM hydrogel or precursor thereof. As used herein, “exogenous” polymer means a polymer not present in the amnionic membrane used to prepare the AM hydrogel or precursor thereof.

The AM may be enzymatically solubilized prior to hydrogel or precursor fbrmation. In this embodiment, any suitable enzyme may be used to solubilize the AM, including but not limited to pepsin or any other protease.

The AM may he present in the hydrogel or precursor thereof at any suitable concentration. In sonic embodiments, the AM is present at between about 1 mg/ml and about 15 mg/ml, about 1 mg/ml and about 14 mg/ml, about 1 mg/ml and about 13 about 1 mg/ml and about 12 mg/ml, about 1 mg/ml and about 11 mg/ml, about 1 mg/ml and about 10 mg/ml, about 1 mg/ml and about 9 mg/ml, about 1 mg/ml and about 8 mg/ml, about 2 mg/ml and about 15 mg/ml, about 2 mg/ml and about 14 mg/ml, about 2 mg/ml and about 13 mg/ml, about 2 mg/ml and about 12 mg/ml, about 2 mg/ml and about 11 mg/ml, about 2 mg/ml and about 10 mg/ml, about 2 mg/ml and about 9 mg/ml, or about 2 mg/ml and about 8 mg/ml in the hydrogel. In a specific embodiment, the AM is present at between about 2 mg/ml and about 8 mg/ml in the hydrogel.

Any suitable buffer may be used to dilute the AM to a desired concentration in the resulting hydrogel or precursor thereof. In non-limiting embodiments, the buffer may comprise physiological saline, phosphate-buffered saline, with or without serum albumin (such as bovine serum albumin (BSA)).

The AM hydrogel may be of any shape or dimensions as deemed appropriate for an intended use. In various embodiments, the hydrogel or precursor thereof can be used in a range of volumes, including but not limited to a range of between about 50 μl and about 50 ml. In some embodiments, the AM may be mixed with different polymers including but not limited to hyaluronan, chitosan, alginate, collagen, dextran, pectin, carrageenan, polylysine, gelatin and/or agarose. In one embodiment, no detectable exogenous polymer is present in the AM hydrogel or precursor thereof.

In another embodiment, the AM hydrogel has a swelling ratio of between about 5% and about 15%. The swelling ratio is a measure of the gel (and not the solution) before and after adding buffer, such as PBS. The ability of AM hydrogels to swell helps govern the diffusion of oxygen and nutrient required for cell growth in the AM hydrogel.

In various embodiments, the hydrogel has a storage modulus of between about 100 Pa to about 10,000 Pa, about 100 Pa to about 9,000 Pa, about 100 Pa to about 8,000 Pa, about 100 Pa to about 7,000 Pa, about 100 Pa to about 6,000 Pa, about 100 Pa to about 5,000 Pa, about 100 Pa to about 4,000 Pa, about 100 Pa to about 3,000 Pa, or about 100 Pa to about 2,000 Pa.

In another embodiment, the hydrogel has shear-thinning properties. As used herein, “shear-thinning” refers to decrease in the complex viscosity with increase in shear rate. The shear thinning property of the AM hydrogels facilitate easy delivery through, for example, a syringe or catheter.

As disclosed herein, the AM hydrogels can be used, for example, in cell delivery and for tissue regeneration in a minimally invasive and cost-effective manner. Thus, in another embodiment, hydrogels may further comprise biological cells within the hydrogel. Any suitable biological cells may be used in the hydrogels of the disclosure. In one embodiment, the biological cells comprise stem cells. The stem cells may be of any suitable type, including but not limited to human or animal adult stem cells, embryonic stem cells and induced pluripotent stem cells. In one embodiment, the stem cells comprise mesenchymal stem cells or adipose-derived stem cells. In all embodiments, the cells may be from human sources (for use in human applications, for example) or from animal sources (for use in veterinary applications, for example). The cells may be naturally occurring or may be engineered in any suitable way as appropriate for an intended use. The biological cells in the hydrogel may be all of one type, or combinations of cell types as suitable fir an intended use.

Any suitable concentration of biological cells may be present in the hydrogel as suitable for an intended use. In various embodiments, the biological cells are present at a concentration of between about 1×10⁵ cells/m1 and about 1×10⁸ cells/ml, about 1×10⁵ cells/ml and about 5×10⁷ cells/ml, or about 1×10⁵ cells/ml and about 1×10⁷ cells/ml.

In another embodiment, the disclosure provides pharmaceutical compositions comprising the AM hydrogel or precursor thereof of any embodiment or combination of embodiments of the disclosure and a pharmaceutically acceptable carrier. Any suitable pharmaceutically acceptable carrier may be used as appropriate for an intended use, including but not limited to physiological buffer. The pharmaceutical composition may be formulated for any suitable route of administration, including topical, spray, drop, and by injection. When formulated for injection, the composition, such as a composition comprising AM hydrogel precursor, may be present with an injection device, including but not limited to a syringe, or a catheter. The pharmaceutical compositions may comprise any other therapeutic component as deemed appropriate for an intended use, including but not limited to other cell types, secretomes, nano/micro particles, proteins and peptides, small molecular weight drugs, growth factors etc.

In another aspect, the disclosure provides methods for treating a disorder, comprising administering to a subject in need thereof an amount effective to treat the disorder of the AM hydrogel, precursor thereof, or pharmaceutical composition of any embodiment or combination of embodiments disclosed herein. As disclosed above, the AM hydrogels are capable of gelation at physiological pH and temperature and thus can form a gel upon injection to a subject, such as at a joint. The AM hydrogels or precursors thereof can be used, for example, in cell delivery and for tissue regeneration in a minimally invasive and cost-effective manner. The methods may comprise the ment of any disorder that can suitably be treated using the AM hydrogel, precursor thereof, or pharmaceutical composition of any embodiment or combination of embodiments disclosed herein. in various embodiments, the disorder may be selected from the group consisting of an inflammatory disease, inflammatory and degenerative conditions of the soft tissues and joints, a musculoskeletal tissue order, a skin tissue disorder including but not limited to burns, wounds, and ulcers; and an eye disorder including but not limited to a corneal defect. In other embodiments, the disorder comprises an inflammatory andlor degenerative conditions of the soft tissues or joints including but not limited to plantar fasciitis, achilles tendinosis, joint tendinitis, tennis/golfer's elbow, ligament damage, arthritis including but not limited to osteoarthritis and rheumatoid arthritis, rotator cuff inflammation and/or degeneration, and discogenic pain, in one embodiment, the disorder comprises osteoarthritis.

As used here, a subject “in need thereof” refers to a subject that has the disorder to be treated or is predisposed to or otherwise at risk of developing the disorder.

As used here, the terms “treatment” and “treating” means:

(i) inhibiting the progression of the disorder;

(ii) prophylactic use for example, preventing or limiting development of a disorder in an individual who may be predisposed or otherwise at risk to the disorder but does not yet experience or display the pathology or symptomatology of the disorder;

(iii) inhibiting the disorder; for example, inhibiting a disorder in an individual who is experiencing or displaying the pathology or symptomatology of the disorder; and/or

(iv) ameliorating the referenced disorder, for example, ameliorating a disorder in an individual who is experiencing or displaying the pathology or symptomatology of the disorder (i.e., reversing or improving the pathology and/or symptomatology) such as decreasing the severity of the disorder.

As used herein, the term “subject” refers to any animal, including mammals such as mice, rats, other rodents, rabbits, dogs, cats, swine, horses, livestock (e.g., pigs, sheep, goats, cattle), primates or humans. In one embodiment, the subject is a human subject.

The administering may be via any suitable route of administration, including topical and by injection. The may comprise administering any other therapeutic component as deemed appropriate for an intended use.

In another aspect, the disclosure provides methods for preparing a decellularized and enzymatically solubilized amnionic membrane (AM) hydrogel, comprising:

(a) decellularizing amniotic membrane by application of a strong base, including but not limited to NaOH, to produce decellularized AM;

(b) enzymatically soluhilizing the decellularized AM to produce a decellularized and enzymatically solubilized amnionic AM;

(c) diluting the decellularized and enzymatically solubilized amnionic AM to a desired concentration and pH in buffer to produce a decellularized and enzymatically solubilized amnionic AM solution; and

(d) heating the decellularized and enzymatically solubilized amnionic AM solution to form a decellularized and enzymatically solubilized amnionic AM hydrogel.

As described in detail in the examples that follow, the decellularization method and hydrogel preparation disclosed herein is simpler and less time consuming compared to other hydrogel methods where decellularization takes several days and solubilization takes 72 hours. The benefits of the hydrogels of the disclosure are detailed above.

The AM can be from any suitable source as noted above, such as human or animal placenta. The step of decellularizing amniotic membrane by application of the strong base may comprise the use of any suitable concentration of strong base, such as about 0.1M to about 0.5 M strong base. In various non-limiting embodiments, the strong base may comprise one or more of lithium hydroxide (LiOH), sodium hydroxide (NaOH), potassium hydroxide (KOH), rubidium hydroxide (RbOH), cesium hydroxide (Cs0H), calcium hydroxide (Ca(OH)₂), strontium hydroxide (Sr(OH)₂), and barium hydroxide (Ba(OH)₂). In one embodiment, the strong base is NaOH. This step can be carried under any suitable conditions, including but not limited to carrying out at about room temperature.

In one embodiment, the step of enzymatically solubilizing comprises contacting decellularized AM with pepsin or other suitable protease wider conditions and for a time suitable to promote enzymatic solubilization of the decellularized AM.

In one embodiment, the method further comprises lyophilizing the decellularized AM prior to step (b).

The step of heating may comprise heating the decellularized and enzymatically solubilized amnionic AM at between about 20° C. and about 40° C., between about 20° C. and about 37° C., between about 25° C. and about 40° C., between about 25° C. and about 37° C., or about 37° C. for a time sufficient to form the hydrogel. In one embodiment, the heating step comprises heating at about 37° C.

In various embodiments, the AM is diluted to be present in the hydrogel and/or the decellularized and enzymatically solubilized amnionic AM solution at between about 1 mg/ml and about 15 mg/ml, about 1 mg/ml and about 14 mg/ml, about 1 mg/ml and about 13 mg/ml, about 1 mg/ml and about 12 mg/ml, about 1 mg/ml and about 11 mg/ml, about 1 mg/ml and about 10 mg/ml, about 1 mg/ml and about 9 mg/ml, about 1 mg/ml and about 8 mg/ml, about 2 mg/ml and about 15 mg/ml, about 2 mg/ml and about 14 mg/ml, about 2 mg/ml and about 13 mg/ml, about 2 mg/ml and about 12 mg/ml, about 2 mg/ml and about 11 mg/ml, about 2 mg/ml and about 10 mg/ml, about 2 mg/ml and about 9 mg/ml, or about 2 mg/ml and about 8 mg/ml,

In another embodiment, the methods further comprise adding biological cells to the decellularized and enzymatically solubilized amnionic AM prior to step (d). In one embodiment, the decellularized and enzymatically solubilized amnionic AM solution is maintained on ice and cells are added prior to the heating step. Any suitable cells may be added, as disclosed above. In one embodiment, the biological cells comprise stem cells, including but not limited to human or animal adult stem. cells, embryonic stem cells and induced pluripotent stem cells. In another embodiment, the stem cells comprise mesenchymal stem cells or adipose-derived stem cells. In a further embodiment, the biological cells are added at a concentration of between about 1×10⁵ cells/ml and about 1×10⁸ cells/ml, about 1×10⁵ cells/ml and about 5>10⁷ cells/ml, or about ×10⁵ cells/ml and about 1×10⁷ cells/ml of the decellularized and enzymatically solubilized amnionic AM solution.

### EXAMPLES

### Materials and Methods

### AM Tissue Collection and Decellularization

Discarded, de-identified placental tissue was collected from the UCONN Health, Connecticut. Amnion and chorion layers were mechanically delaminated from the fresh placental tissue. Under sterile conditions harvested AM were washed with normal saline containing 100 units/mL of penicillin and 100 μg/mL of streptomycin to remove blood and mucus. Next, the membrane was decellularized using 0.5M NaOH followed by two cycles of rinsing the membrane with sterile DI water. Decellularized AM was then lyophilized and stored at room temperature. The decellularization process was verified by PI staining for DNA which examined for the absence of nuclei. Briefly, AM tissue sections were fixed in acetone, rehydrated, rinsed in water, stained for 10 min, and then rinsed with PBS and stored in the dark.

### Biochemical Quantification

The biochemical composition DNA, glycosaminoglycan (GAG), and its collagen contents) of native and decellularized AM were quantified using various biochemical quantification methods. The tissues were lyophilized and 1 mg of dry AM tissue was digested with 3 U/ml papain at 60° C. before analysis. The measured values were normalized to the dry weight of the samples.

### DNA Quantification

DNA was isolated and quantified using a Quant-iT PicoGreen dsDNA assay kit following manufacturer's instructions. Briefly, after papain digest, DNA samples were mixed with the Quant-iT PicoGreen reagent, measured via spectrophotometry at 535 nm with excitation at 485 nm, and DNA content was quantified using a standard curve and normalized to dry AM tissue weight.

### Glycosaminoglycan Quantification

To directly measure the sulfated glycosaminoglycan (GAG) content, papain-digested samples were stained with 1,9-dimethylmethylene blue (DMB, Sigma) and photometrically measured at 525 nm as described. A dilution series of chondroitin sulfate in PBS was used as the standard solution.[31]

### Collagen Quantification

Total collagen was quantified using a hydroxyproline assay, as previously described [32,33]. Tissues were digested in 50 U/mL, papain (Sigma) overnight at 37° C., and then incubated in 6 N HCl at 1.10° C. for 20 hours. The samples were dried and analyzed using a hydroxyproline assay kit (Sigma) and hydroxyproline as a standard. The collagen content was normalized to dry weight of AM tissue.

### AM Hydrogel Preparation and Characterization

### Preparation of AM Hydrogels

To develop an injectable form of AM, the decellularized AM was first lyophilized, ground into a coarse powder and solubilized enzymatically. AM was digested in a porcine pepsin solution in a ratio of 10:1 (AM:Pepsin). Pepsin was used at a concentration of 1 mg/ml using 0.01N HCl. The mixture was then stirred at room temperature for 48 hours. The AM digests were neutralized to a pH of 7.4 by adding 0.1M NaOH, followed by 10× PBS to stop the pepsin activity and provide isotonic solution. The neutralized AM was then diluted to the desired final AM concentration (8 mg/ml, 6 mg/ml, 4 mg/ml and 2 mg/ml) with 1× PBS on ice. The AM pre-gel with different concentration were then placed in an incubator heated to 37° C. to form hydrogels. AM pre-gels were then injected through a syringe to determine injectability.

### Equilibrium Swelling Ratio Analysis

Swelling ratios of the AM gel (8 mg/ml, 6 mg/ml, 4 mg/ml, 2 mg/ml) were measured gmvitnetrically. After gelation, the samples (n=4 per group) were incubated in PBS (pH=7.4) at 37 C for 24 h, in order to measure their wet weight at maximum saturation. The swelling ratios of different gels were calculated using the following equation: swelling ratio (%)=(Ww−Wi)/ Ww×100, where Ww and Wi are the weights of the hydrogels in the equilibrium swelling state and initial gelling state, respectively.

Degradation of AM gels in PBS and Collagenase

The degradation of AM gel (8 mg/ml, 6 mg/ml, 4 mg/ml, 2 mg/ml) was studied in two types of degradation solutions 1 ml of PBS (pH 7.4) with or without collagenase (10 U/ml, Gibco). The AM gel (8 mg/ml, 6 mg/ml, 4 mg/ml, 2 mg/ml) were equally weighed, immersed in 10 ml degradation media, and continuously oscillated at 37° C. Gels in PBS were removed after 0, 1, 5, 10, 15, 30 days, whereas gels in collagenase-containing medium were collected after 5 hours, day 1, 5, 7. Finally, all of the gels collected at different time points were freeze-dried and weighed as Wt. In addition, the dry weight of gels obtained at day 0 was noted as initial weight (Wi). The degradation of the gel was calculated using the following formula:

Degradation %=Wi−Wt/Wi×100

### Rheological Measurements

The rheological measurements of the AM gel (8 mg/ml, 6 mg/ml, 4 mg/ml, 2 mg/ml) and were carried out by rheometer. The sample was placed between parallel plates with a diameter of 20 mm and a gap of 1 min. For time sweeping tests, the storage moduli G′ and of the gel was monitored as functions of time at a frequency of 1 Hz and a stress strain of 1% under a constant temperature of 37 C. To assess the viscosity, steady shear sweep analysis of various AM pre-gels were performed at a constant temperature of 15 C

### Fourier Transform Infrared Spectroscopy

Fourier transform infrared (FTIR) spectroscopy was performed to confirm the presence of characteristic AM bands. Different concentration of AM gels were prepared, and then frozen and lyophilized. Infrared spectra for AM gels were measured by ATR-FTIR in the spectral range of 4000-500 cm-1 using an accumulation of 264 scans with a resolution of 4 cm-1. A background scan was obtained in the absence of material, and the baseline was normalized for each sample after acquisition.

### In Vitro Cell Culture Experiments

### Adipose Derived Stem Cell (ADSCs) Isolation and Characterization

ADSCs were isolated from SD rats 6-8 weeks around 250 grams. Rats were euthanized with CO2 inhalation and neck dislocation. Dead rats were weighed, shaved and cleaned with 70% Ethanol (3 liters). Inguinal fat pads were isolated and weighed. The fat pads were collected and washed thrice in sterile HBSS with 1% antibiotic-antimycotic followed by mincing the tissues into small pieces. Equal volume of collagenase (0.1%) in IIBSS was added to the fat tissue and agitated at 37 C for 90 minutes. The cell suspension was filtered through 100 μm filter (BD Falcon, USA) for the removal of the solid aggregates The collagenase was neutralized by adding equal volume of DMEM-F12 with 10% FBS and 1% antibiotic-antimycotic. The mixture was centrifuged at 1500 rpm for 10 minutes and 2 ml of red cell lysis buffer was added to the pellet and incubated for 2 minutes. The mixture was centrifuged again at 1500 rpm for 10 minutes. Supernatant was discarded and cells were counted and plated in T25/T75 flask containing DMEN-F12 with 10% FBS and 1% antibiotic antimycotic Media was changed after 24 hours. Next, they were cultured for 2 weeks according to standard procedures in DMEM-F12 supplemented with 1% penicillin/streptomycin. ADSCs reaching 80-90% confluence were detached with 0.25% trypsin (Sigma-Aldrich) for 5 min at room temperature and then re-plated.

ADSCs were characterized by using cell surface markers by fluorescence-activated cell sorting (FACS) analysis at passage 3. Cells were characterized Passage 2, 3. ADSCs were detached with 0.5% trypsin-EDTA (Gibco) at 37° C. for approximately 5 min and centrifuged at 1500 rpm for 10 min. Cells were washed using sterile PBS, centrifuged and re-suspended in sterile FACS buffer (PBS, 1% FBS) containing 10 μl of the FITC-conjugated CD29 antibody, FITC conjugated CD90 antibody, FITC conjugated 105. FITC conjugated CD45, PE conjugated CD11b for 30 min. Unlabeled cells were used as controls. Cells were then scanned with FACS and cells were acquired and gated using forward scatter (FSC) and side scatter (SSC) parameters to exclude cell debris and aggregates.

ADSC Encapsulation with AM Gels

The ADSCs were encapsulated in different concentration of AM gels by using the following method. The cells were trypsinized using 0.25% trypsin-EDTA at passage 3. The isolated ADSCs were mixed with different concentration of AM solution to form cell-hydrogel constructs containing 1×106 cells per mL of hydrogel. The cell suspension mixed with pre-gel solution, was slowly dropped into well plates and allowed to gel at a 37° C. 5% CO2 incubator. After gelation (about 10 minutes for all concentrations) the wells were filled with DMEM/F12 containing 10% FBS, 1% pen/strp for up to 7 days. Cells were seeded on tissue culture plate (TCP) and considered as control group.

### ADSC Viability and Proliferation

The LIVE/DEAD assay was used to visualize the distribution of living and dead cells in the hydrogel at different time points. The gels were washed in PBS and incubated with 4 mM calcein-AM and 2 mM ethidium homodimer-I at 37° C., washed in PBS, and imaged by fluorescent methods within 1 h. Live cells were stained green because of the cytoplasmic esterase activity, and dead cells were stained red by ethidium, which enters the cells via damaged cell membranes and becomes integrated into the DNA strands Fluorescence images were taken using confocal microscope (Zeiss LSM 880 Confocal Microscope). The cell proliferation with AM gels and TCP was detected by Ki-67 immunostaining, a pmliferation marker. The gels were washed with PBS followed by fixing in cold 100% Methanol for 5 minutes, pemieabilized with 0.5% Triton-X for 30 min followed by incubation with blocking buffer (2% bovine serum albumin) for 1 hour. The gels were incubated with primary antibody for Ki67 (1:100 dilution) for lh followed by incubation with secondary antibody for Ki67 (Alexa Fluor 488 goat anti-rabbit, Invitrogen, 1:200 dilution). The gels were then stained with PI for 30 minutes. The gels were imaged using confocal microscopy (Zeiss LSM 880 confocal Microscope).

The metabolic activity, viability and proliferation of cells in the hydrogels was assessed using the CellTiter® 96 AQueous nonradioactive cell proliferation assay (MIS assay; Promega) following the manufacturer's protocol. Briefly, the gels were collected in a new plate and washed with PBS. MTS reagent in a ratio of 5:1 (media:MTS) was added to each well at each time point. The plates were then incubated for 4 h in an incubator at 37° C. and 5% CO2. The absorbance of the resulting solution was read at 490 nm using a microplate reader.

Cell proliferation was detected by using assay PicoGreen fluorescent DNA quantification (Molecular Probes) kit at days 1, 4, 7 for both AM gels and TCP. The gels were washed with PBS and digested with collagenase I (0.1%) at 37° C. for 60 minutes. DNA samples were mixed with the Quant-iT PicoGreen reagent, measured via spectrophotometry at 535 nm with excitation at 485 nm and compared with a DNA standard curve provided with the kit. The cell proliferation within the gels and plate was further confirmed by flow cytometry. The cells were isolated from the gels and plate, suspended in PBS and counted using MACSQUFANT analyzer (Miltenyi Biotech)

ADSC Sternness within AM Gels

ADSCs at P3 were encapsulated within the different concentration of AM gels as previously described. After day 1, 4 and 7 the gels were collected in a new plate and washed with PBS. For flow cytometry analysis, the gels were digested with collagenase I (0.1%) for 60 minutes at 37 C, followed by washing the cell pellet in PBS. Cells were stained with 10 μl of the FITC-conjugated CD29 antibody, FITC conjugated CD90 antibody, FITC conjugated 105, FITC conjugated CD45, PE conjugated. CD11b, FITC conjugated CD31 and PE conjugated CD34 for 30 min. Unlabeled cells were used as controls. Cells were then scanned with FACS and cells were acquired and gated using forward scatter (FSC) and side scatter (SSC) parameters to exclude cell debris and aggregates.

Quantitative real-time PCR was employed to determine the ADSC sternness within the gels and TCP. Total RNA was isolated using the RNeasy Mini Kit according to the manufacturer's instructions. For cDNA synthesis, 2 μg total RNA was used as a template for Sprint RT Complete cDNA synthesis kit (Clontech, Mountain, Calif.) in a total volume of 20 μL. For quantitative real-time PCR, iCycler Thermal Cycler Base (Bio-Rad, Hercules, Calif.) and iQ Supermix (Bio-Rad), Sox-2. Oct-4 and GAPDH gene probes were used. Threshold cycle values of target genes was standardized against GAPDH expression and normalized to the expression in the control culture. The fold change in expression was calculated using the ΔΔCt comparative threshold cycle method.[34]

### Anti-Inflammatory and Immunosuppressive Effects of ADSC and AM Gels

### In Vitro Primary Chondrocyte Culture

Rat chondrocytes were obtained from Articular Engineering. Primary culture of chondrocytes was performed using rat articular chondrocytes which were plated in tissue flasks at a density of 10,000-20,000 cells per cm². The chondrocytes were maintained with chondrocyte growth medium containing 10% serum and 5 ug/ml gentamicin. When cells were subconfluent, they were detached by sequential treatment with 0.25% trypsin/EDTA.

### Co-Cculture of ADSCs and Chondrocytes

To investigate the paracrine effect of ADSCs on chondrocytes, we used a co-culture system. Chondrocytes were plated into 24-well plates at a density of 1×10⁵/well and cultured for 24 hours. The culture medium was then refreshed with DMEM/F12 containing 20 ng/mL IL-1β to induce chondrocyte inflammatory responses for a further 24 h in the experimental group. IL-1β treated chondrocytes were cultured with transwell inserts containing 1×10⁵ /well ADSCs (Group 3), 100 μl AM gel (6 mg/ml) (Group4) and 100 μl AM gel (6 mg/ml) (Group5) with ADSCs. The cells were cultured in 2 mL DMEM/F12 supplemented with 5% FBS, 1% penicillin/streptornycin and 20 ng/mL IL-1β. Chondrocytes not treated with IL-1β served as a positive control (Group1). Chondrocytes treated with 20 ng/ml IL-1β served as Group2. After culturing for 72 hours days, the chondrocytes were tested by MTS for viability and nitric oxide assay using NO assay kit and RT-PCR. The concentration of NO was measured by the Griess reagent according to manufacturer's instruction. The media from each group was collected and treated with Griess reagent in room temperature for 30 min. Then the samples were measured in a microplate reader at 570 nm. Gene expression studies were done as described above with the following primers: MMP3, MMP13, ADANTTS-5, IL-6 and TIMP.

### Results

### Design and Characterization of Amnion Gels

### AM Processing and Decellularization

AM was mechanically separated from the human placental tissue obtained from UCONN Health followed by thorough cleaning with saline and sterile water. The AM tissue was then decellularized using 0.5M NaOH to reliably remove amniotic cells from the membrane. We evaluated the decellularization process by PI staining to detect residual DNA on the AM tissue,

The PI fluorescence DNA dye staining of decellularized AM tissue samples showed no detectable DNA residues. In contrast, the control aroup (AM tissue without decellularization) showed substantial amounts of DNA, indicating the presence of AM epithelial cells.

### Biochemical Characterization

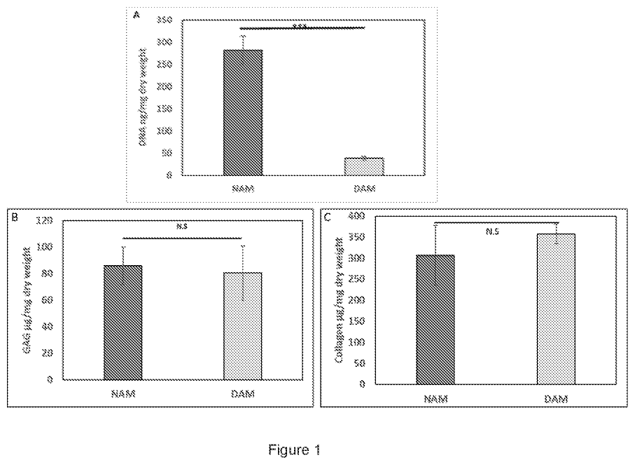

The results from PI staining on decellularization was confirmed by quantifying the DNA content of both native and decellularized. AM using PicoGreen assay. The native AM tissue showed 281.0±31.8 μg of DNA per mg dry weight in comparison to decellularized AM which contained only 38.8±4.2 μg of DNA per mg dry weight strengthening our previous decellularization data (FIG. 1A). In order to evaluate whether the decellularization process affected the Matrix components, we quantified the glycosaminoglycan and total collagen content in both native and decellularized AM. The native AM contained 86.09±14.1 μg GAGs per mg of dry tissue weight whereas the decellularized AM showed 80.4±20.6 μg GAGs per mg of dry tissue weight. There was no sianificant difference in the GAG content of the native and decellularized AM tissue, indicating that our decellularization treatment did not have much effect on the GAG content. (FIG. 1B) To determine the collagen preservation after decellularization, hydroxyproline quantification assay was performed. The results showed that native AM contained 306±71.9 μg collagen per mg of dry tissue weight and the decellularized AM showed 357±22.9 μg collagen per mg of dry tissue weight. (FIG. 1C) No significant differences have been observed in the collagen content between the groups indicating no loss of collagen after the decellularization process.

### Preparation of AM Gel

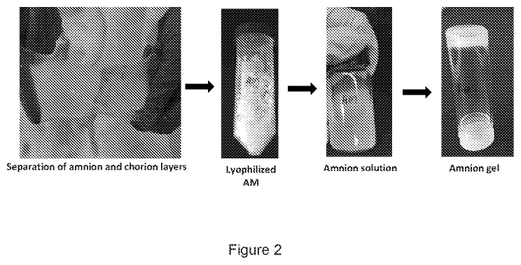

The preparation process of the AM gel is presented in FIG. 2. After decellularization, lyophilized AM was digested enzymatically to solubilize the AM. Solubilized AM was then neutralized, followed by gelation induced at 37° C. for 5-10 minutes. The gels were successfully prepared from AM matrix at various concentrations 8 mg/ml, 6 mg/ml, 4 mg/ml, 2 mg/ml and were found to be injectable through a syringe. Concentrated gels (8 mg/ml, 6 mg/ml) were found to be more rigid macroscopically, compared to the less concentrated aels which were weaker and difficult to handle,

### Physical Properties of AM Gels

### Swelling Ratio and Degradation Properties

The swelling ratio of the AM gels 8 mg/ml, 4 mg/ml, 2 mg/ml was measured in PBS gravimetrically. The AM gels showed a swelling ratio ranging from 5% to 15%. The swelling ratio was found to increase with the increasing concentration of the gel.

Degradation studies for the AM gels were carried out in PBS (hydrolysis) or 10 U/ml collagenase I (enzymolysis) at 37° C. by measuring their weight losses. The degradation in PBS showed moderate degradation up to 30 days, with the degradation rate increasing with the decreasing concentration of the gels. After 30 days, AM 8 mg/ml showed only about 33% degradation, followed by AM 6 mg/ml (39%), 4 mg/ml (42%) and 2 mg/ml (49%). In comparison, enzymatic degradation resulted in faster and severe degradation up to 7 days. Degradation was found to be faster in low concentration gels with 90% degradation in AM 2 mg/ml followed by AM 4 mg/ml (80%), 6 mg/ml (78%) and 8 mg/ml (68%) after 7 days treatment. The weight loss rate also suggested the steady breakdown of AM gel.

### Rheological Properties

The rheological characteristics of AM gels were determined using parallel plate rheometer. The storage modulus (G′) was found to change over time characterized by a sigmoidal curve shape after the temperature of the sample was raised from 10° C. to 37° C. The modulus of gels was found to increase after the AM pre gel solution was neutralized and the temperature increased from 10 to 37° C. The gelation rate increased with increasing AM concentration. The modulus (Pa) were calculated to be AM 8 mg/ml (1643 Pa), AM 6 mg/ml (871 Pa), AM 4 mg/ml (287 Pa), AM 2 mg/ml (126 Pa) respectively. The viscosity of the AM gels with different concentration were then measure over a range of shear rates. All the different concentration AM gels showed similar linear profiles when plotted on a log-log scale, with a decrease in the complex viscosity as the shear rate increased indicating that they are shear thinning. Shear thinning property is very important for translation of an injectable hydrogel as highly viscous or shear thickening material may block the catheter.

### FTIR Spectroscopy

FT-IR spectral analysis showed characteristic bands of amnion membrane. Being a collagenous tissue, AM tissue at all concentration revealed 3 characteristic absorption bands at 1652 cm-1 (amide I, C═O stretching), 1550 cm-1 (amide bending), and 1339 cm-1 (amide III, N—H bending). The spectra also exhibited peaks at 3315 and 2936 cm-1, assigned to the stretching vibrations of N—H and C—H bonds, respectively. The peaks at 1454 cm-1 signify the —C—O stretching present in the —O—COCH3 group and those observed at 1082 cm-1 correspond to the aliphatic chain with a primary amino group. The AM gel contained at 1240 cm-1 indicative of S═O stretch of R—SO3⁻¹ characteristic of polysaccharides from 1200 to 1000 cm-1. [35,36]

### Amnion Based Hydrogel Can Support ADSC Survival and Proliferation

ADSC Characterization and Encapsulation within AM Gels

Adipose derived stem cells were successfully isolated from rat inguinal fat pads. After 1 day of culture, spindle shaped cells were found to be attached to the T flasks. Primary ADSC culture took 5˜6 days to reach confluence. ADSCs at passage 3 were analyzed for expression of MSC specific cell-surface markers. ADSCs were found to be negative for the hematopoietic lineage marker CD45 with 6.5%, and negative for CD11b (0.2%). ADSCs were found to be positive for CD29 (98.2%), CD90 (98.6%) and CD105 (85%), These results are consistent with previously published studies [14], ADSCs (P3) at a concentration of 1×10⁶ cells/ml of AM were encapsulated within different concentrations of AM gels 8 mg/ml, 6 mg/ml, 4 mg/ml and 2 mg/ml and cultured for up to 7 days in DMEM/FI2 containing 10% FBS, 1% Pen/strp. The cells within the gels were characterized at regular time periods of day 1, day 4 and day 7.

In Vitro ADSC Viability within AM Gels

The biocompatibility of the hydrogels with ADSCs was evaluated by live/dead assay. To investigate the cell adhesion and survival, rat ADSCs (P3) were encapsulated in hydrogel scaffold at a final concentration of 1×10⁶cells/ml for up to 7 days. ADSCs seeded on tissue culture plate was considered to be the control group. The LIVE/DEAD® assay was used to visualize the distribution of live and dead cells after 1, 4 and 7 days in the 3D cell culture system. Calcein AM stains live cells and fluoresces green upon the reaction of intracellular esterase whereas ethidium hotnodimer-I stains dead cells red. The gels at all concentration showed presence of viable cells mainly with few dead cells after day 1,4,7 indicating that the cell viability was maintained within the AM gels. ADSCs readily adhered to the surface of AM gels and were found to be stretched with spindle-like shape on TCP as well as all the AM gels. The successful adherence and spreading of ADSCs to the AM gels was due to the binding domains present within AM.

### In Vitro ADSC Proliferation Within AM Gels

The ADSC proliferation was evaluated quantitatively by MTS assay, DNA quantification, and flow cytometry and qualitatively by Ki-67 staining Ki-67 protein, which is associated with cell proliferation, was found to be expressed by ADSCs encapsulated within all the gels after 4 and 7 days of cultureas well as on TCP suggesting that cells cultured in hydrogel have proliferative ability.

The MTS assay was utilized to quantify the viability and proliferation of encapsulated ADSCs after day 1, 4 and 7 which revealed that all the concentration of AM gels supported ADSC viability and proliferation. Cell proliferation was fliund to increase in AM 8 mg/ml, 6 mg/ml, 4 mg/ml from day 1 to day 7. However, cell proliferation in AM 2 mg/ml was found to reduce on day 7. The cell proliferation was found to be highest in TCP followed by AM 8 mg/ml, AM 6 mg/ml, 4 mg/ml, 2 mg/ml after all time points. There was no significant difference in cell proliferation between TCP and AM 8 mg/ml and AM6 mg/ml. Cell proliferation by MTS assay was further corroborated by DNA quantification and cell count by flow cytometry. DNA quantification and flow cytometry analysis revealed similar pattern of cell proliferation as observed by MTS assay. Cell proliferation increased from day 1 to day 7 in AM 8 mg/ml, 6 mg/ml, 4 mg/ml except for AM 2 mg/ml. The reason for decrease in proliferation from high to low concentration of AM gel is due to the shrinking of gels. Shrinking of gel at lowest concentration (AM 2 mg/ml) is maximum caused by the contractile forces generated by the ADSCs. Shrinking of gel results in maximum cell migrating out of the gel to the plate. Shrinking was found to be maximum in AM 2 mg/ml followed by AM 4 mg/ml. There was not much shrinking observed in AM 6 mg/ml and AM 8 mg/ml. In order to verify that decrease in cell proliferation in lower concentration AM gels is due to gel shrinking, we counted the number of cells within the gels and the plate by flow cytometry. It was further confirmed that cell are migrating out of the gel due to shrinking and this is why cell proliferation by MTS assay and DNA quantification was found to decrease in AM 4 mg/ml and AM 2 mg/ml. We can thus conclude that the gels are biocompatible and can provide a suitable microenvironment for maintain the ADSC viability up to 7 days.

### Amnion Based Hydrogel Can Support ADSC Sternness.

Flow Cytometry Analysis to Determine Sternness of ADSCs within AM Gels

To further investigate the changes in the ADSC characteristics within the AM gels, we performed flow cytometry and gene expression analysis. Flow cytometry analysis revealed the presence of positive and absence of negative CD markers on ADSCs encapsulated within AM gels. The ADSCs within all the AM gels were found to be positive for stem cell markers, CD90 and CD 29 after day 1, day 4, day 7. Moreover, they were found to be negative for other markers, CD45, CD11b, CD31 and CD34 after day 1, day 4, day 7 of culture.

Gene Expression Analysis to Determine Sternness of ADSCs within AM Gels

The flow cytometry analysis was confirmed by the gene expression analysis which revealed that ADSCs encapsulated within all the AM gels showed higher expression of Sox2 and OCT4 compared to the TCP group, However, hydrogels with higher concentration exhibited higher Sox2 and Oct4 expression compared to lower concentration AM groups. The gene expression of Sox2 and OCT 4 was found to decrease with decreasing concentration with highest expression in AM 8 mg/ml followed by AM 6 mg/m, AM4 mg/ml and AM 2 mg/ml at all time points.

### Evaluating the Anti-Inflammatory and Immunosuppressive Effects of ADSC and Amnion Based Gels.

We next aimed at studying the most important phase of this study, which is to investigate the paracrine effect of ADSCs within the gel and synergistic role of AM with ADSCs to inhibit the catabolic response of inflamed chondrocytes. The experimental groups were divided into 5 groups-Group1: chondrocyte alone (control), Group2: chondrocytes with IL-1β (20 ng/mL), Group3: chondrocytes with IL-1β (20 ng/mL) and ADSCs, Group4: chondrocytes with IL-1β (20 ng/mL), and AM (6 mg/ml) and Group5: chondrocytes with IL-1β (20 ng/mL) and ADSCs encapsulated within AM (6 mg/ml) gel. After 24 h, cell viability of the chondrocytes was determined using the MIT assay and compared among the three groups.

MTS Assay and Nitric oxide (NO) Assay

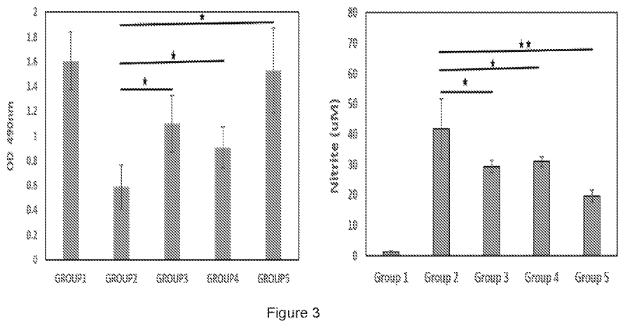

After 24 hours of culture, cell viability was measure via MTS assay. Cell viability was found to reduce upon IL-1β (20 ng/mL) treatment compared to control group. The addition of ADSCs (group3), AM gel (group4) and AM gel with ADSCs (group5) were found to significantly increase the cell viability of chondrocytes treated with IL-1β (20 ng/mL). In addition, group5 (ADSC within AM gel) showed highest cell viability, indicating the synergistic effect of AM gel and ADSC in preventing the effect of pro-inflammatory cytokine. (FIG. 3A)

During inflammatory process, NO is generated by activated iNOS which induce intracellular signal transduction and inflammatory gene activation. NO has also been shown to inhibit synthesis of collagens and proteoglycans and increase MMP activity. Our study showed that IL-1β (20 ng/mL) treatment increased NO production in group 2 compared to the control group. The treatment with ADSCs (group3), AM gel (group4) and AM gel with ADSCs (group5) were found to significantly reduce the NO production in chondrocytes (group 2) treated with IL-1β (20 ng/mL). Similar to MTS assay, we observed that group5 AM gel with ADSC showed lowest NO production compared to the group3 (ADSC) and group4 (AM gel), confirming the synergistic effect of gel and cell. (FIG. 3B)

### Gene Expression Analysis to Evaluate Effect of ADSCs and AM Gels on Cytokine Treated Chondrocytes

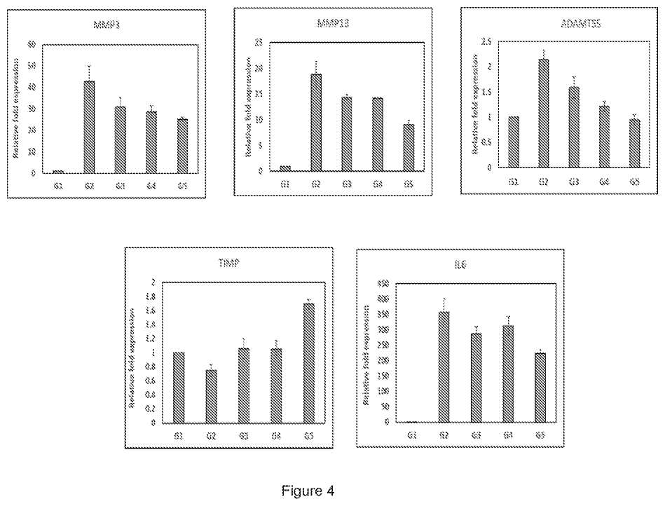

The IL-1β-treated group induced chondrocyte catabolism by increasing the mRNA expression levels of the matrix-degrading enzymes MMP-3, MMP-13, ADAMTS5 and the pro-inflammatory cytokines IL-6, compared to those in the untreated control group. ADSCs, AM gel, AM gel with ADSCs were found to decrease the gene expression levels of matrix-degrading enzymes (MMPs, ADAMTS5) and inflammatory factors in chondrocytes treated with IL-1β (20 ng/mL). The treatment groups, group3 (ADSC), group 4 (AM gel) and group5 (AM with ADSC) were found to attenuate the increased mRNA level of IL6, MMP-3, MMP-13 and ADAMTS-5 in chondrocytes caused by IL-1β. The expression of TIMP was found to reduce upon treatment with IL-1β. However, the treatment groups group3 (ADSC), group 4 (AM gel) and group5 (AM with ADSC) were found to increase TIMP expression in IL-1β-treated cells. (FIG. 4) Thus our results confirmed the immunosuppressive effect of ADSCs and upon encapsulation within AM gel further enhanced the immunosuppressive and anti-inflammatory effect, thereby reducing the catabolic response in chondrocyte.

### Intra-Articular Injection of ADSCs in Amnion Based Injectable Hydrogel in a Collagenase Induced OA Rat Model

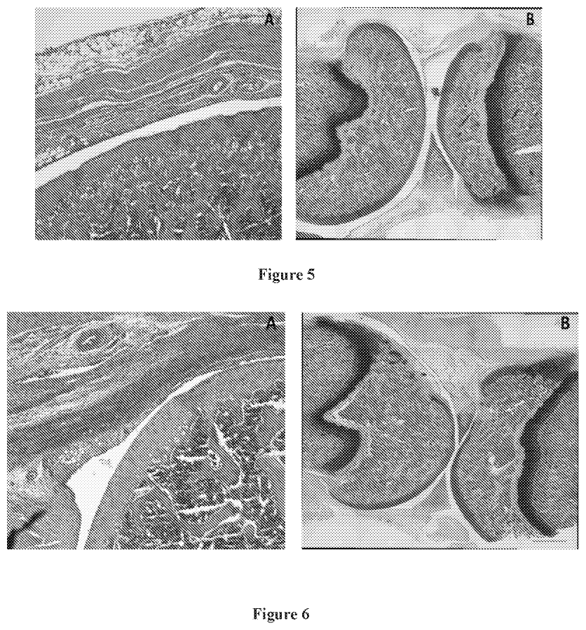

Osteoarthritis was induced by two intra-articular injections of 500 U of collagenase II into the right knee joint of Sprague Dawley rats for up to 1 week. FIG. 5A,B shows H&E staining and Safranin-O staining of sham group with only saline injection and FIG. 6A,B shows animal group, which received collagenase injection up to 1 week. No histopathological damage was noticed in the knee joint of saline-injected sham (control) group after 1 week. The sham group showed no signs of inflammation and cartilage loss as indicated by the H&E staining and Saf-O staining (FIG. 5A,B).

On the other hand, after 1 week of collagenase injection, loss of proteoglycan staining (Safranin-O) was observed as well as a high degree of synovial inflammation with thinning of the articular cartilage was noticed. An increase in the number of synovial lining cell layers and infiltration of inflammatory cells was also observed. Collagenase induced the degeneration of the articular cartilage by directly digesting collagen from the extracellular matrix of cartilage (FIG. 6A,B).

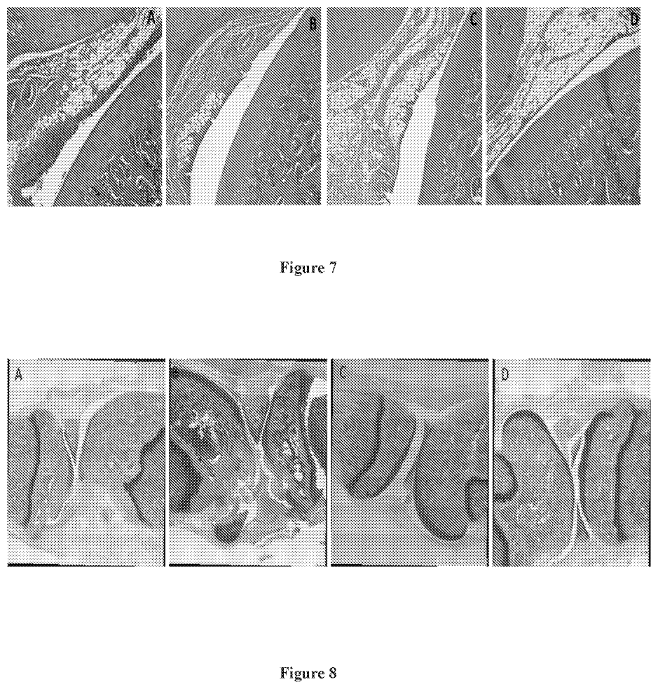

After 1 week of collagenase injection, rats were divided into 4 groups: Group1: control (PBS), Group 2: ADSC, Group 3: AM gel (6 mg/ml), Group 4: AM gel (6mg/ml) with ADSCs. The animals were sacrificed after 4 weeks of treatment. PBS treated animals at 4 weeks showed pronounced synovial inflammation with an increase in number of synovial lining cell layers (FIG. 7A), lesions and areas of erosion were prominent along with diminished Saf-O staining of both femoral and tibial surface suggesting loss of proteoglycan content (FIG. 8A). ADSC treatment reduced the synovial inflammation (FIG. 7B), and preserved the loss of proteoglycan content of cartilage ECM to some extent, but the lesion and areas of erosion are still evident (FIG. 8B). In contrast, histological analysis of AM gel (FIG. 7C, 8C) and AM gel with ADSC (FIG. 7D,8D) treated joints showed significant reduction in synovial inflammation and a smooth cartilage surface with no lesions and strong Saf-O staining. However, the Saf-O staining is more prominent in AM gels with ADSC group compared to only AM group. The in vivo study thereby corroborates the in vitro results and demonstrated the unique synergistic chondroprotective effect of ADSCs and AM gel.

### Discussion

In this study, human amniotic membrane was effectively decellularized using a fast and more efficient method of NaOH treatment. Previous decellularization methods are tedious, long and have adverse effects on tissue architecture and the ECM composition. To overcome these problems, we decellularized AM by gently rubbing a cotton swab soaked in NaOH over the AM which leads to nearly complete and easy removal of adherent cells in less than a minute. Complete decellularization of a tissue minimizes potentially negative immune responses, host acceptance and improved tissue remodeling outcome. Quantifying DNA content is considered to be a potential indicator of remaining cell debris and decellularization degree. Our DNA content in the decellularized AM was below the threshold of 50 ng/mg dry ECM weight decellularized biomaterials. The decellularization method did not show much effect on the collagen and GAG content of AM matrix.

The physical properties showed that AM hydrogels are capable of swelling up to 15% and thus may govern the diffusion of oxygen and nutrient required for cell growth in and out of the hydrogel. The AM gels were found to he degradable by natural process, which is another important factor needed for proper tissue growth and remodeling. AM gels were found to undergo faster gelation which is desired for in vivo applications to limit the cell loss from the site of application. The gelation temperature is for self-assembling hydrogels especially injectable hydrogel, is an important parameter for clinical application, particularly their ability to gel at body temperature. Once processed, the AM is a free flowing liquid and upon neutralizing the salt and pH and increasing the temperature to 37° C. it forms into a gel. The AM eels showed shear-thinning property which is again very important while considering a material for delivery via injection. The shear thinning property of AM gels would facilitate easy delivery through syringe/catheter.

Next, we investigated the biocompatihility of AM gels with ADSCs. We found that AM gels were able to support ADSC viability, arowth and proliferation within the gels. ADSCs successfully adhered to the amnion hydrogel matrix, exhibited their normal morphology and proliferated at rates comparable to conventional 2D culture. Further, it was found that AM gel also maintained the sternness properties of the ADSCs. It is believed that the therapeutic properties of MSCs are derivative of their paracrine activity, thus it is important to maintain the sternness properties of ASDCs.

Finally, our results showed the immunosuppressive and anti-inflammatory properties of ADSC along with the AM. This is a very important finding which may have potential role as a therapeutic for treating critical disease such as osteoarthritis. It is known that inflammation and its induced catabolism plays an important role during osteoarthritis by increasing MMPs, and destructing the ECM. ADSCs are known to possess immune-suppressive and chondro-protective properties. ADSCs secrete multiple immunosuppressive factors, like IL-10, IL-1 Receptor Antagonist (IL-1RA), TGFβ and induce anti-inflammatory effects in macrophages. ADSCs can also decrease inflammation by changing the macrophage phenotype from M1 (classically activated) to M2 (alternatively activated). The delivery of ADSCs in an AM gel would further add up to the prevention of inflammation. Thus, we explored the effect of ADSCs when delivered via injectable AM hydrogel on inhibiting the catabolic responses in chondrocytes using a transwell system without a direct contact. In this study, the inflammatory condition was induced by treating rat chondrocytes with IL-1β, a condition close to the inflammatory environment occurring in the cartilage during OA[22]. Upon treating the inflamed chondrocytes with ADSC, AM gel and AM with ADSCs, the inflammatory responses along with matrix degrading enzymes were reduced. Our study confirmed the synergistic role of AM and ADSCs in suppressing the inflammation in chondrocytes and thus might play important cartilage protective roles during osteoarthritis.

This injectable AM hydrogel system can thus be used in both basic research and clinical application leading to a major breakthrough in stem cell therapeutics

### REFERENCES

1. Johnson V L, Hunter D J2. The epidemiology of osteoarthritis, Best Pra.ct Res Clin Rheumatol. 2014; 28(1):5-15.
2. Kong L, Zheng L Z, Chin L, Ho K K W. Role of mesench, nal stern cells in osteoarthritis treatment, J Orthop Translat, 2017; 9:89-103.
3. Glyn-Jones S, Palmer A J, Agricola R, Price A J, Vincent T L, H, et al. Osteoarthritis. Lancet. 2015; 386:376-387
4. Pelletier J. P. Martel-Pelletier J. Abramson S. B. Osteoarthritis, an inflammatory disease: potential implication for the selection of new therapeutic targets. Arthritis Rheum. 2001; 44:1237.
5. Smith M. D. Triantafillou S. Parker A. Youssef P. P. Coleman M. Synovial membrane inflammation and cytokine production in patients with early osteoarthritis. J Rheumatol. 1997; 24:365.
6. Bondeson J. Wainwright S. D. Lauder S. Amos N. Hughes C. E. The role of synovial macrophages and macrophage-produced cytokines in driving aggrecanases, matrix metalloproteinases, and other destructive and inflammatory responses in osteoarthritis. Arthritis Res Ther. 2006; 8:R187.
7. Ollivierre F, Gubler U. Towle C. A. Laurencin C. Treadwell B. V. Expression of IL-1 genes in human and bovine chondrocytes: a mechanism for autocrine control of cartilage matrix degradation. Biochem Biophys Res Commun. 1986; 141:904
8. Loeser R. F, Goldring S. R, Scanzello C. R, Goldring M. B. Osteoarthritis: a disease of the joint as an organ. Arthritis Rheum, 2012; 64:1697-1707.
9. Liu-Bryan R, Terkeltaub R. Emerging regulators of the inflammatory process in osteoarthritis. Nat Rev Rheumatol. 2015; 11:35-44.
10. Dave, M. and Amin, A R Yin-Yang regulation of prostaglandins and nitric oxide by PGD2 in human arthritis: reversal by celecoxib, Immunol. Lett. 2013; 152:47-54.
11. Fu Y, Lei J, Zhuang Y, Zhang K, and Lu D. Overexpression of HMGB1 A-box reduced IL-1beta-induced MMP expression and the production of inflammatory mediators in human chondrocytes. Exp. Cell Res. 2016; 349:184-190.
12. Murab S. Chameettachal S, Bhattacharjee M, Das S, Kaplan D L. Ghosh S. Matrix-embedded cytokines to simulate osteoarthritis-like cartilage microenvironments. Tissue Eng Part A. 2013; 19(15-16):1733-53.
13. Escobar J L, Bhattachaijee M, Kuyinu E, Nair L S, Laurencin C T. Regenerative Engineering for Knee Osteoarthritis Treatment: Biomaterials and Cell-Based Technologies. Engineering. 2017; 3(1):16-27.
14. Musculoskeletal Tissue Regeneration: the Role of the Stem Cells. Regenerative Engineering and Translational Medicine, 2017; 3(3):133-165.
15. Close J. E, Browne T. P. Branch Surgical alternatives for treatment of articular cartilage lesions. J Am Acad Orthop Surg. 2000; 8(3):180-189.
16. Steinwachs M, Kreuz P C, Autologous chondrocyte implantation in chondral defects of the knee with a type collagen membrane: a prospective study with a 3-year follow-up. Arthroscopy. 2007; 23(4):381-387.
17. Kon E, Filardo G, Gobbi A, Berruto M, Andriolo L, Ferrua, et al. Long-term results after hyaluronan-based NL&CT for the treatment of cartilage lesions of the patellofemoral joint. Am J Sports Med. 201;44 (3):602-608.
18. Mehrabani D, Mehrahani G, Zare S, Manafi A. Adipose-derived stem cells (ADSC) and aesthetic surgery: a mini review. World J Plast Surg. 2013; 2:65-70.
19. Zuk P A, Zhu M, Ashjian P, de Ugarte D A, Huang J I, Mizuno H, et al. Human adipose tissue is a source of .multipotent stem cells. Mol Biol Cell. 2002; 13:4279-4295.
20. Manferdini C, Maumus Gabusi E, Piacentini A, Filardo G, Peyrafitte J A, et al. Adipose-derived mesenchymal stem cells exert antiinflaminatory effects on chondrocytes and synoviocytes from osteoarthritis patients through prostaglandin E2. Arthritis Rheum. 2013; 65:1271-81.
21. Maumus M, Manferdini C, Toupet K, Peyrafitte J A, Ferreira R, Facchini A, Gahusi E, Bourin P, Jorgensen C, Lisignoli G, Noel D. Adipose mesenchymal stein cells protect chondrocytes from degeneration associated with osteoarthritis. Stem Cell Res. 2013; 11(2):834-44.
22. Jiang; L B, Lee S, Wang Y, Xu Q T, Meng; D H, Zhang J. Adipose-derived stem cells induce autophagic activation and inhibit catabolic response to pro-inflammatory cytokines in rat chondrocytes. Osteoarthritis Cartilage. 2016; 24(6):1071-81.
23. Desando G, Cavallo C, Sartain F, Martini I, Parrilli. A, Veronesi. F, et al. Intra-articular delivery of adipose derived stromal cells attenuates osteoarthritis progression in an experimental rabbit model. Arthritis Res Ther. 2013; 15:R22.
24. Toghraie F, Pazinkhah M, Gholipour M A, Faghih Z, Chenari N, Nezhad S, et al. Scaffold-free adiposederived stem cells (ASCs) improve experimentally induced osteoarthritis in rabbits. Arch Iran Med. 2012; 15:495±499.
25. Vilar J M, Batista N I, Morales N I, Santana A, Cuervo B, Rubio N I, Cugat R, Sopena J, Carrillo T M. Assessment of the effect of intraarticular injection of autologous adipose-derived mesenchymal stem cells in osteoarthritic dogs using a double blinded force platform analysis BMC BMC Vet Res. 2014; 10:143.
26. C. H. Jo, Y. G. Lee, W. H. Shin, H. Kim, J. W. Chai, E. C. Jeong, et al. Intra-articular injection of mesenchymal stem cells for the treatment of osteoarthritis of the knee: a proof-of-concept clinical trial. Stem Cells 2014; 32(5):1254-1266.
27. Koh Y G, Choi Y J, Kwon S K, Kim Y S, Yeo J E. Clinical results and second-look arthroscopic findings after treatment with adipose-derived stein cells for knee osteoarthritis. Knee Sura Sports Traumatol Arthrosc. 2015; 23(5):1308-1316.
28. Rana D, Tabasuma A, Ramalingam N I. Cell-laden alginate/polyacrylamide beads as carriers for stein cell delivery: preparation and characterization. RSC Adv. 2016; 6:20475.
29. Yuan X, Wei Y, Villasante A, Ng J J D, Arkonac D E, Chao P G, Vunjak-Novakovic G. Stem cell delivery in tissue-specific hydrogel enabled meniscal repair in an orthotopic rat model. Biomaterials. 2017; 132:59-71.
30. Niknejad H, Peirovi H, Jorjani M, Ahmadiani A, Ghanavi J, Seifalian A M. Properties of the amniotic membrane for potential use in tissue engineering. Eur Cell Mater. 2008; 15:88-99.
31. Bhattachatjee M, Miot S, Gorecka A, Singha K, Loparic Ni, Dickinson S, Das A, Bhavesh N S, Ray A R, Martin I, Ghosh S. Oriented lamellar silk fibrous scaffolds to drive cartilage matrix orientation: towards annulus fibrosus tissue engineering. Acta Biomater. 2012; 8(9):3313-25.
32. Bhattacharjee M, Chawla S, Chameettachal S, Murab S, Bhavesh N S, Ghosh S. Role of chondroitin sulphate tethered silk scaffold in cartilaginous disc tissue regeneration. Biomed Mater. 2016; 11(2):025014.
33. Bhattacharjee Chameettachal S, Pahwa S, Ray A R, Ghosh S. Strategies for replicating anatomical cartilaginous tissue gradient in engineered intervertebral disc. M Bhattacharjee, S Chameettachal, S Pahwa, A R Ray, S Ghosh. ACS Appl Mater Interfaces. 2014; 6(1):183-93.
34. Bhattachaijee N I, Schultz-Thater E, Trella E, Miot S, Das S, Loparic N I, Ray A R, Martin I, Spagnoli G C, Ghosh S. The role of 3D structure and protein conthrmation on the innate and adaptive immune responses to silk-based biomaterials. Biomaterials, 2013; 34(33):8161-71.
35. Kumar T R, Shanmugasundaram N, Babu M. Biocompatible collagen scaffolds from a human amniotic membrane: physicochemical and in vitro culture characteristics. J Biomater Sci Polym Ed. 2003; 14(7):689-706.
36. Camacho N P, West P, Torzilli P A, Mendelsohn R. FTIR microscopic imaging of collagen and proteoglycan in bovine cartilage. Biopolymers. 2001; 62(1):1-8.
37. Saghizadeh M, Winkler M A, Kramerov A A, Hetnmati D M, Ghiam C A, Dimitrijevich S D, Sareen D, Ornelas L, Ghiasi H, Bninken W J, Maguen E, Rabinowitz Y S, Svendsen C N, Jirsova. K, Ljubimov A V. A Simple Alkaline Method for Decellularizing Human Amniotic Membrane for Cell Culture. PLoS One. 2013; 8(11):79632.
38. Reing J E, Brown B N, Daly K A, Freund J M, Gilbert T W, Hsiong S X, Huber A, Kullas K E, Toney S, Wolf M T, Badylak S F, The effects of processing methods upon mechanical and biologic properties of porcine dermal extracellular matrix scaffolds. Biomaterials. 2010; 31:8626-8633.
39. Murphy M B, Moncivais K, Caplan A I. Mesenchymal stem cells: environmentally responsive therapeutics for regenerative medicine. Exp Mol Med. 2013; 45:54.
40. Solomon A. Rosenblatt M, Monroy D, Ji Z, Pflugfelder S C, Tseng S C. Suppression of interleukin lalpha and interleukin lbeta in human limbal epithelial cells cultured on the amniotic membrane stromal matrix. Br J Ophthalmol. 2001; 85(4):444-9.
41. Hao Y, Ma D H, Hwang D O, Kim W S, Zhang F. Identification of antiangiogenic and anti-inflammatory proteins in human amniotic membrane. Cornea. 2000; 19(3):348-52.
42. Buhimschi I A. Jahr M, Buhimschi C S. Petkova A P, Weiner C P, Saed G M, The novel antimicrobial peptide beta3-defensin is produced by the amnion: a possible role of the fetal membranes in innate immunity of the amniotic cavity. Am J Obstet Gynecol. 2004; 191(5):1678-87.
43. King A E, Paltoo A, Kelly R W, Sallenave J M, Bocking A D, Challis J R. Expression of natural antimicrobials by human placenta and fetal membranes. Placenta Placenta. 2007; 28(2-3):161-9.

1. A decellularized amnionic membrane (AM) hydrogel, or a precursor thereof.

2. The AM hydrogel or precursor thereof of claim 1, wherein no detectable exogenous polymer is present in the hydrogel.

3. The AM hydrogel or precursor thereof of claim 1, wherein the AM is present at between about 1 mg/ml and about 15 mg/ml, about 1 mg/ml and about 14 mg/ml, about 1 mg/ml and about 13 mg/ml, about 1 mg/ml and about 12 mg/ml, about 1 mg/ml and about 11 mg/ml, about 1 mg/ml and about 10 mg/ml, about 1 mg/ml and about 9 mg/ml, about 1 mg/ml and about 8 mg/ml, about 2 mg/ml and about 15 mg/ml, about 2 mg/ml and about 14 mg/ml, about 2 mg/ml and about 13 mg/ml, about 2 mg/ml and about 12 mg/ml, about 2 mg/ml and about 11 mg/ml, about 2 mg/ml and about 10 mg/ml, about 2 mg/ml and about 9 mg/ml, or about 2 mg/ml and about 8 mg/ml in the hydrogel or precursor thereof.

4. The AM hydrogel or precursor thereof of claim 1, wherein the hydrogel has a swelling ratio of between about 5% and about 15%.

5. The AM hydrogel or precursor thereof of claim 1, wherein the hydrogel has a storage modulus of about 100 Pa to about 10,000 Pa and/or has shear-thinning properties.

6. The AM hydrogel or precursor thereof of claim 1, further comprising biological cells within the hydrogel or precursor thereof.

7-10. (canceled)

11. A pharmaceutical composition comprising:
(a) the AM hydrogel or precursor thereof of claim 1; and
(b) a pharmaceutically acceptable carrier.

12. (canceled)

13. A method for treating a disorder, comprising administering to a subject in need thereof and amount effective to treat the disorder of the AM hydrogel or precursor thereof of claim 1.

14. The method of claim 13, wherein the disorder is selected from the group consisting of an inflammatory disease, inflammatory and degenerative conditions of the soft tissues and joints, a musculoskeletal tissue order, a skin tissue disorder including but not limited to burns, wounds, and ulcers; and an eye disorder including but not limited to a corneal defect.

15-16. (canceled)

17. A method for preparing a decellularized amnionic membrane (AM) hydrogel, comprising:
(a) decellularizing amniotic membrane by application of a strong base, including but not limited to NaOH to produce decellularized AM;
(b) enzymatically solubilizing the decellularized AM to produce a decellularized and enzymatically solubilized amnionic AM;
(c) diluting the decellularized and enzymatically solubilized amnionic AM to a desired concentration and pH in buffer to produce a decellularized and enzymatically solubilized amnionic AM solution; and
(d) heating the decellularized and enzymatically solubilized amnionic AM solution to form a decellularized and enzymatically solubilized amnionic AM hydrogel.

18. The method of claim 17, wherein the strong base is applied at a concentration of about 0.1M to about 0.5M.

19. The method of claim 17, further comprising lyophilizing the decellularized AM prior to step (b).

20. The method of claim 17, wherein the enzymatically solubilizing comprises contacting decellularized AM with pepsin under conditions and for a time suitable to promote enzymatic solubilization of the decellularized AM.

21. The method of claim 17, wherein the heating comprises heating the decellularized and enzymatically solubilized amnionic AM at between about 20° C. and about 40° C., between about 20° C. and about 37° C., between about 25° C. and about 40° C., between about 25° C. and about 37° C., or about 37° C. for a time sufficient to form the hydrogel.

22. The method of claim 17, wherein the AM is present at between about 1 mg/ml and about 15 mg/ml, about 1 mg/ml and about 14 mg/ml, about 1 mg/ml and about 13 mg/ml, about 1 mg/ml and about 12 mg/ml, about 1 mg/ml and about 11 mg/ml, about 1 mg/ml and about 10 mg/ml, about 1 mg/ml and about 9 mg/ml, about 1 mg/ml and about 8 mg/ml, about 2 mg/ml and about 15 mg/ml, about 2 mg/ml and about 14 mg/ml, about 2 mg/ml and about 13 mg/ml, about 2 mg/ml and about 12 mg/ml, about 2 mg/ml and about 11 mg/ml, about 2 mg/ml and about 10 mg/ml, about 2 mg/ml and about 9 mg/ml, or about 2 mg/ml and about 8 mg/ml in the hydrogel and/or the decellularized and enzymatically solubilized amnionic AM solution.

23. The method of claim 17, further comprising adding biological cells to the decellularized and enzymatically solubilized amnionic AM prior to step (d).

24. The method of claim 23, wherein the biological cells comprise stem cells, including but not limited to human or animal adult stem cells, embryonic stem cells and induced pluripotent stem cells.

25. The method of claim 24, wherein the stem cells comprise mesenchymal stem cells or adipose-derived stem cells.

26. The method of claim 23, wherein the biological cells are added at a concentration of between about 1×105 cells/ml and about 1×108 cells/ml, about 1×105 cells/ml and about 5×107 cells/ml, or about 1×105 cells/ml and about 1×107 cells/ml of the decellularized and enzymatically solubilized amnionic AM solution.

27. The method of claim 23, wherein the biological cells comprise human cells.
